(19) United States
(12) Patent Application Publication
(10) Pub. No.: US 2017/0240951 A1
(43) Pub. Date: Aug. 24, 2017

(54) Detecting Xanthan Gum

(71) Applicant: Saudi Arabian Oil Company, Dhahran (SA)

(72) Inventor: Yara Ahmed Alzahid, Dhahran (SA)

(21) Appl. No.: 15/436,197

(22) Filed: Feb. 17, 2017

Related U.S. Application Data
(60) Provisional application No. 62/299,193, filed on Feb. 24, 2016.

(51) Int. Cl. C12Q 1/34

(52) U.S. Cl. CPC C12Q 1/34; C12Y 302/01006; C12Y 302/01074; G01N 2333/942

(57) ABSTRACT

Detecting xanthan gum in a sampling location includes delivering a tagged polypeptide to the sampling location. The tagged polypeptide includes a polypeptide and a fluorescent probe bound to the polypeptide, such that the fluorescent probe is released from the polypeptide to yield an unbound fluorescent probe when the polypeptide interacts with xanthan gum. Light that excites the unbound fluorescent probe is directed toward the sampling location, and an intensity of fluorescence emitted from the unbound fluorescent probe is assessed, wherein a non-zero intensity is indicative of the presence of xanthan gum in the sampling location. A device for the detection of xanthan gum has a sensing region including the tagged polypeptide, a light source adapted to direct light to the sensing region, the light source adapted to provide one or more wavelengths of light to excite the fluorescent probe, and a detector for detecting fluorescence emitted from the fluorescent probe.

### CROSS-REFERENCE TO RELATED APPLICATION

This application claims the benefit of U.S. application Ser. No. 62/299,193 entitled “DETECTING XANTHAN GUM” and filed on Feb. 24, 2016, which is incorporated herein by reference in its entirety.

### TECHNICAL FIELD

This invention relates to devices, methods, systems, and compositions used to detect the presence of xanthan gum, quantify the presence of xanthan gum, or both, particularly in petroleum production operations.

### BACKGROUND

Xanthan gum is an exocellular biopolymer secreted by Xanthomonas sp. It is a heteropolysaccharide with a primary structure of repeated pentasaccharide units formed by two glucose units, two mannose units, and one glucuronic acid unit. The main chain consists of β-D-glucose units linked at the 1 and 4 positions. The side chain is a trisaccharide, consisting of α-D-mannose that contains an acetyl group, β-D-glucuronic acid, and a β-D-mannose terminal unit, linked to a pyruvate group. The molecular weight distribution can range from 2×10⁶ Da to 20×10⁶ Da, depending on the association between polysaccharide chains (e.g., aggregates of several individual chains) and variations in fermentation conditions.

### SUMMARY

Provided in this disclosure is a xanthan gum detecting system that includes a polypeptide bound to a fluorescent probe, such that the fluorescent probe is released from the polypeptide when the polypeptide contacts xanthan gum.

In some embodiments, the system further includes a light source adapted to provide a wavelength of light that excites the fluorescent probe.

In some embodiments, the fluorescent probe fluoresces when it is released from the polypeptide and the detecting system is configured to detect the presence of xanthan gum in response to an increase in fluorescence. In some embodiments, the fluorescent probe fluoresces when it is bound to the polypeptide and the detecting system is configured to detect the presence of xanthan gum in response to a decrease in fluorescence.

The system can further include a detector for detecting a wavelength of light emitted from the fluorescent probe when the fluorescent probe is excited.

The fluorescent probe can be covalently bound to the enzyme. In some embodiments, the fluorescent probe is a fluorescent carbon nanotube. In some embodiments, the fluorescent probe is a fluorophore. In some embodiments, the fluorophore is Cy®3, Cy®5, methyl blue, methylene blue, or 8-anilinonaphthalene-1-sulfonic acid.

In some embodiments, the polypeptide is an enzyme. In certain embodiments, the polypeptide is β-glucanohydrolase I or β-glucanohydrolase II.

Also provided in this disclosure is a polypeptide bound to a fluorescent probe for the detection of xanthan gum.

In some embodiments, the polypeptide is an enzyme. In certain embodiments, the polypeptide is β-glucanohydrolase I or β-glucanohydrolase II.

In some embodiments, the fluorescent probe includes a fluorescent carbon nanotube. In some embodiments, the fluorescent probe includes a fluorophore. Suitable fluorophores 8-anilinonaphthalene-1-sulfonic acid, Cy®3, Cy®5, methyl blue, and methylene blue, or a combination thereof.

Also provided in this disclosure is a device for the detection of xanthan gum. The device includes a sensing region that includes a polypeptide bound to one or more fluorescent probes; a light source adapted to direct light to the sensing region, the light source adapted to provide a wavelength of light that excites the fluorescent probe; and a detector for detecting fluorescence emitted from the fluorescent probe when it is excited by the light source.

In some embodiments, the fluorescent probe is covalently bound to the polypeptide. In some embodiments, the fluorescent probe is non-covalently bound to the polypeptide. In certain embodiments, the fluorescent probe is a fluorescent carbon nanotube or a fluorophore. Suitable fluorophores include Cy®3, Cy®5, methyl blue, methylene blue, and 8-anilinonaphthalene-1-sulfonic acid.

Also provided herein is a method for detecting xanthan gum in a subterranean formation. The method includes placing a polypeptide bound to a fluorescent probe into the subterranean formation; directing light into the subterranean formation, wherein at least a portion of the light is at a wavelength that excites the fluorescent probe; and assessing an intensity of light emitted from the fluorescent probe, where a non-zero intensity of light emitted from the fluorescent probe indicates the presence of xanthan gum. In some embodiments, the method further includes quantifying an amount or concentration of xanthan gum based on an intensity of the light emitted from the fluorescent probe.

In some embodiments, the polypeptide is β-glucanohydrolase I or β-glucanohydrolase II, and the fluorophore is a fluorescent carbon nanotube, Cy®3, Cy®5, methyl blue, methylene blue, or 8-anilinonaphthalene-1-sulfonic acid (ANS).

Advantages of the disclosed systems and methods include specificity for xanthan gum, accuracy exceeding that of methods seeking to establish a relationship between concentration and viscosity of xanthan-containing fluids, and results independent of impurities that may be present.

### BRIEF DESCRIPTION OF THE DRAWINGS

FIG. 1A depicts bonding of a fluorescent probe to a polypeptide. FIG. 1B depicts release of the fluorescent probe when the polypeptide contacts an analyte.

FIG. 2 is a flow chart of an exemplary process of detecting the presence of an analyte with a tagged fluorescent probe.

FIG. 3 depicts and exemplary system for detecting an analyte.

### DETAILED DESCRIPTION

Devices, systems, methods, and compositions are provided herein for the detection of xanthan gum. The xanthan gum is typically dispersed in a fluid, such as an aqueous-based fluid. In some cases, devices, systems, methods, and compositions provided herein can be used for the detection of an analyte other than xanthan gum. Devices, systems, methods, and compositions provided herein can use a polypeptide (such as an enzyme) that is bound to fluorescent probe (such as a fluorophore) for the detection of an analyte (such as xanthan gum). In one embodiment, a fluorescent probe is released from a polypeptide when the polypeptide comes into contact with an analyte. Cleavage of the fluorescent probe from the polypeptide can cause a change in the fluorescent characteristics of the fluorescent probe such that the detection of, or a change in, fluorescence is indicative of the presence of the analyte. Cleavage of the fluorescent probe from the polypeptide can cause a change in the fluorescent characteristics of the fluorescent probe such that the detection of, or a change in, fluorescence can also be used to quantify the amount or concentration of the analyte.

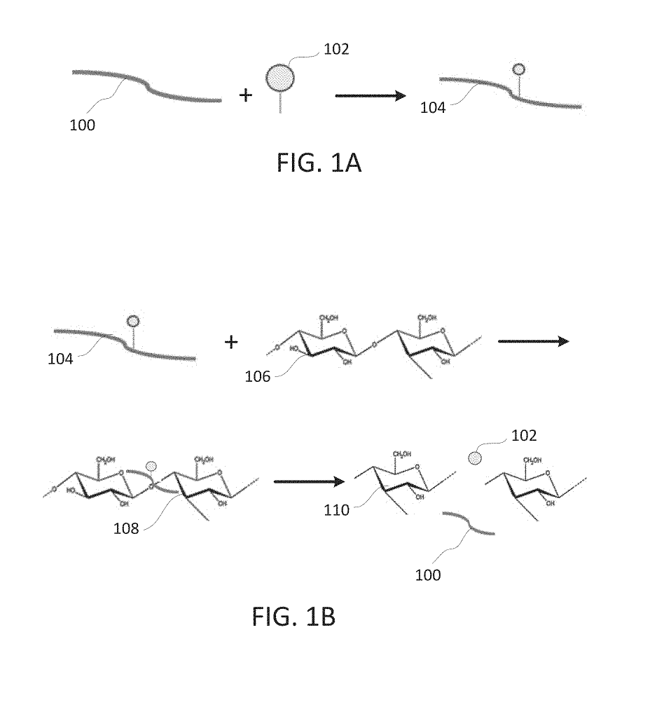

FIG. 1A depicts binding of polypeptide 100 with fluorescent probe 102 to yield tagged polypeptide 104. Fluorescent probe 102 has characteristic excitation and emission wavelengths and thus fluoresces when it is not bound to polypeptide 100. When bound to polypeptide 100, however, fluorescent probe 102 does not fluoresce.

FIG. 1B depicts tagged polypeptide 104 interacting with analyte 106 to yield tagged polypeptide-analyte complex 108. As used herein, “interacting” with analyte 106 includes contacting the analyte, reacting with the analyte, bonding to the analyte, or severing one or more bonds in the analyte, in such a way as to alter one more fluorescence characteristics of the fluorescent probe, resulting in a change in fluorescence that can be detected via ultraviolet-visible (UV-VIS) spectroscopy. The change in fluorescence may be an increase in fluorescence intensity, a decrease in fluorescence intensity, or a shift in fluorescence wavelength. As depicted in FIG. 1B, tagged polypeptide 104 severs a bond in analyte 106 and also releases fluorescent probe 102 from the tagged polypeptide to yield analyte fragments 110 and the fluorescent probe. Fluorescent probe 102, which did not fluoresce when bound to polypeptide 100, fluoresces in its unbound state. Thus, an increase in light detected at an emission wavelength of fluorescent probe 102 indicates the presence of analyte. With other parameters held constant, a greater intensity of emitted light is indicative of a greater amount or concentration of the analyte.

In one example of the process depicted in FIGS. 1A and 1B, the analyte is xanthan gum, and the polypeptide is an enzyme that catalyzes the breakdown of xanthan gum. Exemplary enzymes that catalyze the breakdown of xanthan gum include hydrolases such as β-glucanohydrolase I and β-glucanohydrolase II. Suitable fluorescent probes include fluorescent carbon nanotubes and fluorophores such as Cy®3 (an orange-fluorescent dye, available from ThermoFisher Scientific, excited with 532 nm radiation), Cy®5 (a far-red fluorescent dye, available from ThermoFisher Scientific, excited with 633 nm or 647 nm radiation), methyl blue, methylene blue, a coumarin, tetramethylrhodamine 8-anilinonaphthalene-1-sulfonic acid, 9-anthroylcholine (9-AC), and 5-dimethylaminonaphthalene-5-sulfonic acid (dansyl). The tagged polypeptide may be formed as depicted in FIG. 1A by methods generally known in the art. When the tagged polypeptide is proximate the β-1,4-glucan backbone of xanthan gum, the enzyme hydrolyzes the xanthan gum and the fluorescent probe is released from the enzyme. The fluorescent probe, which does not fluoresce when bound to the enzyme, fluoresces after being released from the enzyme. Fluorescence of the unbound fluorescent probe can be detected via spectroscopy by exciting the fluorescent probe with UV-VIS radiation and assessing an intensity of light emitted from the fluorescent probe. Emission of light from the fluorescent probe indicates cleavage of the fluorescent probe from the enzyme, and thus the presence of xanthan gum. In some cases, an amount or concentration of xanthan gum can be assessed based on an intensity of the light emitted from the released fluorescent probes. That is, with a higher concentration of xanthan gum and an excess of tagged enzymes, more fluorescent probes are released and a greater intensity of light is detected.

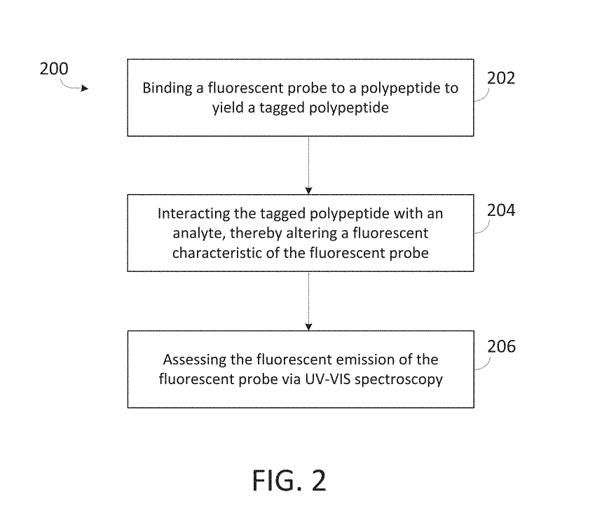

FIG. 2 is a flow chart showing operations in process 200 for detecting the presence of an analyte with a tagged polypeptide. In 202, a fluorescent probe is bound, covalently or otherwise, to a polypeptide to yield the tagged polypeptide. In some embodiments, the polypeptide is an enzyme that cleaves the backbone of xanthan gum, such as β-glucanohydrolase I or β-glucanohydrolase II. Suitable fluorescent probes include fluorescent carbon nanotubes and fluorophores such as Cy®3, Cy®5, methyl blue, methylene blue, a coumarin, tetramethylrhodamine 8-anilinonaphthalene-1-sulfonic acid, 9-anthroylcholine (9-AC), and 5-dimethylaminonaphthalene-5-sulfonic acid (dansyl). In 204, the tagged polypeptide interacts with the analyte, thereby altering a fluorescent characteristic of the fluorescent probe. In one embodiment, when the analyte is xanthan gum and the polypeptide is an enzyme that cleaves the backbone of xanthan gum, interacting with the analyte includes breaking a bond in the analyte and cleaving the fluorescent probe from the enzyme. In 206, fluorescent emission of the fluorescent probe is assessed via UV-VIS spectroscopy. Based on the interaction of the tagged polypeptide with the analyte, fluorescent emission of the fluorescent probe may increase in intensity, decrease in intensity, or shift wavelength.

In some embodiments, an operation in process 200 is omitted. In one example, the tagged polypeptides are obtained prior to implementation of process 200. In some embodiments, process 200 includes operations not shown in FIG. 2. In one example, process 200 includes assessing an amount or concentration of analyte based on the fluorescent emission assessed in 206. Assessing an amount or concentration of analyte may include comparing the assessed fluorescent emission with the fluorescent emission of a known amount or concentration of the analyte.

### Xanthum Gum Detecting System

Provided in this disclosure is a xanthan gum detecting system that includes a polypeptide bound to a fluorescent probe such that the fluorescent probe is released from the polypeptide when the polypeptide interacts with xanthan gum.

In one example, the polypeptide is an enzyme that catalyzes the hydrolysis of xanthan gum and the fluorescent probe is released from the polypeptide when the enzyme catalyzes the hydrolysis of xanthan gum. In some embodiments, the enzyme is β-glucosidase, β-mannosidase, or α-mannosidase. In some embodiments, the enzyme is hydrolase such as β-glucanohydrolase I or β-glucanohydrolase II.

In some embodiments, the fluorescent probe is covalently bound to the polypeptide. In certain embodiments, the fluorescent probe is bound to the polypeptide through a disulfide linkage, an amide linkage, an ester linkage, a carbamate linkage, a thioester linkage, a thioate linkage, a phosphodiester linkage, or a diphosphate linkage. In some embodiments, the fluorescent probe is covalently bound to the polypeptide through a linker. In some embodiments, the fluorescent probe is non-covalently bound to the polypeptide. In certain embodiments, the fluorescent probe is bound to the polypeptide through hydrogen bonding, charge-charge interactions, van der Waals forces, hydrophobic interactions, or a combination thereof

In some embodiments, the detecting system is configured to detect the presence of xanthan gum in response to an increase in fluorescence and the fluorescent probe fluoresces when it is released from the polypeptide and has a lower fluorescence or does not fluorescence when it is bound to the polypeptide. In some embodiments, the detecting system is configured to detect the presence of xanthan gum in response to a decrease in fluorescence and the fluorescent probe fluoresces when it is bound to the enzyme and has a lower fluorescence or does not fluoresce when it is not bound to the polypeptide.

In some embodiments, the fluorescent probe is a fluorescent carbon nanotube. The fluorescent carbon nanotube may fluoresce when it is released from the polypeptide (unbound) and have a lower fluorescence or does not fluoresce when it is bound to the polypeptide. Alternatively, the fluorescent carbon nanotube may fluoresce when it is bound to the polypeptide and does not fluoresce or has a lower fluorescence when it is released from the polypeptide. The fluorescent carbon nanotube can be released from the polypeptide following a conformational change in the polypeptide as a result of the polypeptide interacting with xanthan gum. If the fluorescent carbon nanotube is covalently bound to the polypeptide, the fluorescent carbon nanotube can be released from the polypeptide when the covalent bond breaks as a result of the polypeptide interacting with xanthan gum.

In some embodiments, the fluorescent probe is a fluorophore. Suitable fluorophores include Cy®3 and Cy®5, methyl blue, methylene blue, a coumarin, tetramethylrhodamine 8-anilinonaphthalene-1-sulfonic acid, 9-anthroylcholine (9-AC), and 5-dimethylaminonaphthalene-5-sulfonic acid (dansyl). In certain embodiments, the fluorophore fluoresces in the orange region of the visible spectrum and can be excited with an excitation wavelength of 532 nm and visualized with a tetramethylrhodamine (TRITC) filter set. In other embodiments, the fluorophore fluoresces in the far-red region and can be excited with an excitation wavelength of 633 nm or 647 nm.

In some embodiments, fluorophore fluoresces when it is released from the polypeptide and has a lower fluorescence or does not fluoresce when it is bound to the polypeptide. In some embodiments, the fluorophore fluoresces when it is in contact with the polypeptide and does not fluoresce or has a lower fluorescence when it is released from the polypeptide. The fluorophore can be released from the polypeptide following a conformational change in the polypeptide as a result of an interaction between the polypeptide and xanthan gum. If the fluorophore is covalently bound to the polypeptide, the fluorophore can be released from the polypeptide when the covalent bond breaks as a result of the polypeptide interacting with xanthan gum.

In some embodiments, a xanthan gum detecting system includes a light source adapted to provide a wavelength of light to excite the fluorescent probe. The system can further include a detector for detecting a wavelength of light emitted from the fluorescent probe when the fluorescent probe is excited. For example, the system can include a UV-VIS detector. The detector can include a fluorescence monochromator. The detector can also include a photomultiplier.

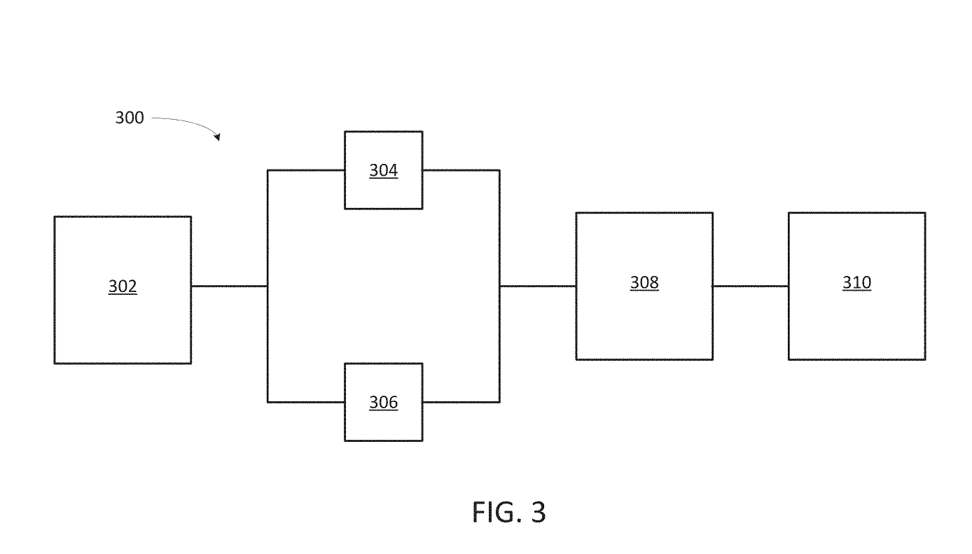

FIG. 3 depicts exemplary system 300 for detecting an analyte, such as xanthan gum, with a tagged polypeptide, such as a tagged enzyme. Light source 302 provides light at a wavelength to a sampling location 304. Light source 302 may include a laser or a broadband UV-VIS lamp. In some embodiments, light from light source 302 is also provided to a reference location 306, to provide a reference beam intensity. In some embodiments, sampling location 304 is a portion of a subterranean formation. Sampling location 304 includes a tagged polypeptide in a fluid. In certain embodiments, the analyte is present in sampling location 304. When the tagged polypeptide and the analyte interact in sampling location 304 such that a fluorescent characteristic of the fluorescent fluorophore is altered, a change in emitted fluorescent intensity or wavelength or the presence of emitted fluorescence is detected by detector 308. In some embodiments, detector 308 includes a photomultiplier. This change in emitted fluorescent intensity is indicative of the presence of the analyte. In some embodiments, the intensity of emitted fluorescence is compared with calibration standards to assess an amount or concentration of analyte in sampling location 304.

In some embodiments, the polypeptide includes a quencher compound. The quencher compound can be a quencher fluorophore. In some embodiments, the quencher compound is a quencher dye. That is, as a distance between the quencher compound and the fluorescent probe decreases, the fluorescence of the fluorescent probe decreases, and as a distance between the quencher compound and the fluorescent probe increases, the fluorescence of the fluorescent probe increases. When the polypeptide interacts with xanthan gum (such as catalyzing the hydrolysis of xanthan gum), the distance between the quencher compound and the fluorescent probe changes, resulting in a change in fluorescence of the fluorescent probe that can be measured by fluorescent resonance energy transfer. In one example, when the polypeptide interacts with xanthan gum, the distance between the quencher compound and the fluorescent probe can decrease, resulting in a decrease in the fluorescence of the fluorescent probe that can be measured by fluorescent resonance energy transfer. Alternatively, when the polypeptide interacts with xanthan gum, the distance between the quencher compound and the fluorescent probe can increase, resulting in an increase in the fluorescence of the fluorescent probe that can be measured by fluorescent resonance energy transfer.

In some embodiments, the fluorescent probe is covalently bound to the N-terminus of the polypeptide and the quencher compound is covalently bound to the C-terminus of the polypeptide. In some embodiments, the fluorescent probe is covalently bound to the C-terminus of the polypeptide and the quencher compound is covalently bound to the N-terminus of the polypeptide.

Also provided in this disclosure is a xanthan gum detecting system that includes an enzyme bound to a fluorescent probe such that the fluorescent probe is released from the enzyme when the enzyme interacts with xanthan gum. Suitable enzymes include β-glucanohydrolase I and β-glucanohydrolase II. Suitable fluorescent probes fluorescent carbon nanotubes, Cy®3, Cy®5, methyl blue, methylene blue, and 8-anilinonaphthalene-1-sulfonic acid. In some embodiments, the system includes a light source adapted to provide a wavelength of light to excite the fluorescent probe and a detector for detecting a wavelength of light emitted from the fluorescent probe when the fluorescent probe is excited.

### Xanthum Gum Probe

Also provided in this disclosure is a xanthan gum probe. The xanthum gum probe includes a polypeptide and a fluorescent probe.

The polypeptide can be an enzyme. The enzyme can be capable of hydrolyzing xanthan gum. For example, the enzyme can include a hydrolase such as β-glucanohydrolase I or β-glucanohydrolase II.

In some embodiments, the fluorescent probe is covalently bound to the polypeptide. In some embodiments, the fluorescent probe is non-covalently bound to the polypeptide. In certain embodiments, the fluorescent probe is bound to the polypeptide through hydrogen bonding, charge-charge interactions, van der Waals forces, hydrophobic interactions, or a combination thereof

In some embodiments, the fluorescent probe is a fluorescent carbon nanotube. The fluorescent carbon nanotube can be configured such that it fluoresces when it is released from the polypeptide and has a lower fluorescence or does not fluoresce when it is bound to the polypeptide. Alternatively, the fluorescent carbon nanotube can be configured such that it fluoresces when it bound to the polypeptide and does not fluoresce or has a lower fluorescence when it is released from the polypeptide. The fluorescent carbon nanotube can be released from the polypeptide following a conformational change in the polypeptide as a result of the polypeptide interacting with xanthan gum. If the fluorescent carbon nanotube is covalently bound to the polypeptide, the fluorescent carbon nanotube can be released from the polypeptide when the covalent bond breaks as a result of the polypeptide interacting with xanthan gum.

In some embodiments, the fluorescent probe is a fluorophore. Suitable fluorophores include Cy®3, Cy®5, methyl blue, methylene blue, and 8-anilinonaphthalene-1-sulfonic acid.

In some embodiments, fluorophore fluoresces when it is released from the polypeptide and has a lower fluorescence or does not fluoresce when it is bound to the polypeptide. In some embodiments, the fluorophore fluoresces when it is in contact with the polypeptide and does not fluoresce or has a lower fluorescence when it is released from the polypeptide. The fluorophore can be released from the polypeptide following a conformational change in the polypeptide as a result of the polypeptide interacting with xanthan gum. If the fluorophore is covalently bound to the polypeptide, the fluorophore can be released from the polypeptide when the covalent bond breaks as a result of the polypeptide interacting with xanthan gum.

In some embodiments, the polypeptide includes a quencher compound. The quencher compound can be a quencher fluorophore. In some embodiments, the quencher compound is a quencher dye. As a distance between the quencher compound and the fluorescent probe decreases, the fluorescence of the fluorescent probe may decrease. As a distance between the quencher compound and the fluorescent probe increases, the fluorescence of the fluorescent probe may increase. When the polypeptide interacts with xanthan gum (such as catalyzing the hydrolysis of xanthan gum), a distance between the quencher compound and the fluorescent probe can change, resulting in a change in fluorescence of the fluorescent probe that can be measured by fluorescent resonance energy transfer. In one example, when the polypeptide interacts with xanthan gum, a distance between the quencher compound and the fluorescent probe can decrease, resulting in a decrease in the fluorescence of the fluorescent probe that can be measured by fluorescent resonance energy transfer. Alternatively, when the polypeptide interacts with xanthan gum, a distance between the quencher compound and the fluorescent probe can increase, resulting in an increase in the fluorescence of the fluorescent probe that can be measured by fluorescent resonance energy transfer.

In some embodiments, the fluorescent probe is covalently bound to the N-terminus of the polypeptide and the quencher compound is covalently bound to the C-terminus of the polypeptide. In some embodiments, the fluorescent probe is covalently bound to the C-terminus of the polypeptide and the quencher compound is covalently bound to the N-terminus of the polypeptide.

Also provided in this disclosure is a xanthan gum probe that includes an enzyme bound to a fluorescent probe such that the fluorescent probe is released from the enzyme when the enzyme contacts xanthan gum (e.g., hydrolyzes xanthan gum). The enzyme is a β-glucanohydrolase I, a β-glucanohydrolase II, or a combination thereof. The fluorescent probe includes a fluorescent carbon nanotube, Cy®3, Cy®5, methyl blue, methylene blue, 8-anilinonaphthalene-1-sulfonic acid, or a combination thereof.

### Device for the Detection of Xanthan Gum

Also provided in this disclosure is a device for the detection of xanthan gum. The device includes a sensing region that includes a polypeptide bound to a fluorescent probe. The device additionally includes a light source adapted to direct light to the sensing region. Further, the light source is adapted to provide one or more wavelengths of light capable of exciting the fluorescent probe. The device also includes a detector for detecting a fluorescence emitted from the fluorescent probe when it is excited by the light source.

The polypeptide can be any polypeptide as described in this disclosure. The fluorescent probe can be any fluorescent probe as described in this disclosure.

### Kit for the Detection of Xanthan Gum

Also provided in this disclosure is a kit for the detection of xanthan gum. The kit includes a polypeptide and a fluorescent probe.

The polypeptide and fluorescent probe can be kept separate in the kit and then mixed prior to use to allow the fluorescent probe to bind to the polypeptide. The polypeptide and fluorescent probe can be kept together in the kit such that the fluorescent probe is bound to the polypeptide.

In some embodiments, the kit includes a light source and a detector. The light source can be adapted to provide one or more wavelengths of light capable of exciting the fluorescent probe. The detector can be configured to detect fluorescence emission from the fluorescent probe.

The polypeptide can be any polypeptide as described in this disclosure. The fluorescent probe can be any fluorescent probe as described in this disclosure.

### Method for Detecting Xanthan

Also provided is a method for detecting xanthan gum in a subterranean formation.

The phrase “subterranean formation” refers to any material under the surface of the earth, including under the surface of the bottom of the ocean. For example, a subterranean formation can be any section of a wellbore and any section of a subterranean petroleum- or water-producing formation or region in fluid contact with the wellbore. Placing a material in a subterranean formation can include contacting the material with any section of a wellbore or with any subterranean region in fluid contact therewith. Subterranean materials can include any materials placed into the wellbore such as cement, drill shafts, liners, tubing, casing, or screens; placing a material in a subterranean formation can include contacting with such subterranean materials. In some examples, a subterranean formation or material can be any below-ground region that can produce liquid or gaseous petroleum materials, water, or any section below-ground in fluid contact therewith. For example, a subterranean formation or material can be at least one of an area desired to be fractured, a fracture, or an area surrounding a fracture, and a flow pathway or an area surrounding a flow pathway, wherein a fracture or a flow pathway can be optionally fluidly connected to a subterranean petroleum- or water-producing region, directly or through one or more fractures or flow pathways.

The phrase “treatment of a subterranean formation” refers to any activity directed to extraction of water or petroleum materials from a subterranean petroleum- or water-producing formation or region, for example, including drilling, stimulation, hydraulic fracturing, clean-up, acidizing, completion, cementing, remedial treatment, abandonment, and the like.

A method for detecting xanthan gum can include placing a polypeptide bound to a fluorescent probe into the subterranean formation. For example, the polypeptide bound to a fluorescent probe can be placed into the subterranean formation using a suitable fluid. The fluid can be an aqueous-based fluid. Examples of suitable aqueous-based fluids include fresh water; saltwater (such as water containing one or more dissolved salts); brine (saturated salt water), seawater; and any combination thereof. In some embodiments, a method for detecting xanthan gum includes directing light into the subterranean formation. The light can include at least one wavelength adapted to excite the fluorescent probe. The method can also include assessing fluorescent emission from the fluorescent probe as an indication of the presence or absence of xanthan gum in the subterranean formation.

In some embodiments, a method for detecting xanthan gum includes collecting a fluid sample from the subterranean formation and combining the sample with a polypeptide bound to a fluorescent probe. The method can also include assessing fluorescent emission from the fluorescent probe as an indication of the presence or absence of xanthan gum in the subterranean formation.

In some embodiments, a method of detecting xanthan gum includes quantifying the amount of xanthan gum based on an intensity of fluorescent emission from the fluorescent probe.

The polypeptide can be any polypeptide as described in this disclosure. The fluorescent probe can be any fluorescent probe as described in this disclosure.

In some embodiments, a method of detecting xanthan gum includes treating a subterranean formation with a polymer flood after the presence or absence of xanthan gum is assessed or after the concentration of xanthan gum is assessed. In certain embodiments, the method includes adjusting an amount of xanthan gum in the polymer flood based on assessing the presence or absence of xanthan gum or determining a concentration of xanthan gum.

In some embodiments, a method of detecting xanthan gum includes delivering a polypeptide bound to a fluorescent probe to a sampling location; directing, to the sampling location, light capable of exciting the fluorescent probe when the fluorescent probe is separated from the polypeptide; and assessing fluorescent emission from the fluorescent probe in the sampling location.

In some embodiments, the sampling location is a subterranean formation. In some embodiments, the polypeptide bound to the fluorescent probe can be delivered to a sampling location using a suitable fluid, such as an aqueous-based fluid. Examples of suitable aqueous-based fluids include fresh water; saltwater (water containing one or more dissolved salts); brine (saturated salt water), seawater; or any combination thereof.

What is claimed is:

1. A method for detecting xanthan gum in a sampling location, the method comprising:
delivering a tagged polypeptide to the sampling location, the tagged polypeptide comprising:
a polypeptide; and
a fluorescent probe bound to the polypeptide, such that the fluorescent probe is released from the polypeptide to yield an unbound fluorescent probe when the polypeptide interacts with xanthan gum;
directing, to the sampling location, light that excites the unbound fluorescent probe; and
assessing an intensity of fluorescence emitted from the unbound fluorescent probe in the sampling location, wherein a non-zero intensity is indicative of the presence of xanthan gum in the sampling location.

2. The method of claim 1, wherein the polypeptide is β-glucanohydrolase I or β-glucanohydrolase II.

3. The method of claim 1, wherein the fluorescent probe is a fluorescent carbon nanotube or a fluorophore.

4. The method of claim 3, wherein the fluorescent probe is a fluorophore, and the fluorophore is an orange-fluorescent dye excited with 532 nm radiation, a far-red fluorescent dye excited with 633 nm or 647 nm radiation, methyl blue, methylene blue, or 8-anilinonaphthalene-1-sulfonic acid.

5. The method of claim 1, wherein the sampling location is a subterranean formation.

6. The method of claim 1, comprising quantifying an amount or concentration of xanthan gum in the sampling location based on the assessed intensity of the fluorescence emitted from the unbound fluorescent probe.

7. A xanthan gum detecting system comprising:
a tagged polypeptide comprising:
a polypeptide; and
a fluorescent probe bound to the polypeptide, such that the fluorescent probe is released from the polypeptide to yield an unbound fluorescent probe when the polypeptide interacts with xanthan gum.

8. The system of claim 7, comprising a light source adapted to provide a wavelength of light that excites the fluorescent probe.

9. The system of claim 7, wherein the unbound fluorescent probe fluoresces upon excitation, and an increase in fluorescence intensity when the polypeptide interacts with xanthan gum is indicative of the presence of xanthan gum.

10. The system of claim 7, wherein the fluorescent probe fluoresces when it is bound to the polypeptide, and a decrease in fluorescence intensity when the polypeptide interacts with xanthan gum is indicative of the presences of xanthan gum.

11. The system of claim 7, comprising a detector for detecting a fluorescent emission from the fluorescent probe.

12. The system of claim 7, wherein the fluorescent probe is covalently bound to the polypeptide.

13. The system of claim 7, wherein the fluorescent probe is a fluorescent carbon nanotube or a fluorophore.

14. The system of claim 13, wherein fluorescent probe is a fluorophore, and the fluorophore is an orange-fluorescent dye excited with 532 nm radiation, a far-red fluorescent dye excited with 633 nm or 647 nm radiation, methyl blue, methylene blue, or 8-anilinonaphthalene-1-sulfonic acid.

15. The system of claim 7, wherein the polypeptide is an enzyme.

16. The system of claim 15, wherein the enzyme is β-glucanohydrolase I or β-glucanohydrolase II.

17. The system of claim 7, comprising xanthan gum.

18. A device for the detection of xanthan gum, the device comprising:
a sensing region comprising tagged polypeptide, the tagged polypeptide comprising:
a polypeptide; and
a fluorescent probe bound to the polypeptide, such that the fluorescent probe is released from the polypeptide to yield an unbound fluorescent probe when the polypeptide interacts with xanthan gum;
a light source adapted to direct light to the sensing region, the light source adapted to provide one or more wavelengths of light to excite the fluorescent probe; and
a detector for detecting fluorescence emitted from the fluorescent probe.

19. The device of claim 18, wherein the polypeptide is an enzyme.

20. The device of claim 19, wherein the enzyme is β-glucanohydrolase I or β-glucanohydrolase II.
